(19) United States
(12) Patent Application Publication
(10) Pub. No.: US 2016/0356030 A1
(43) Pub. Date: Dec. 8, 2016

(54) Airgust Toilet Tank lid Air freshener

(71) Applicant: Ehis K. Inekhomon, Encino, CA (US)

(72) Inventor: Ehis K. Inekhomon, Encino, CA (US)

(21) Appl. No.: 14/731,270

(22) Filed: Jun. 4, 2015

(51) Int. Cl. E03D 9/00; A61L 9/12; A61L 9/14

(52) U.S. Cl. CPC E03D 9/007; A61L 9/14; A61L 9/127; A61L 9/122

(57) ABSTRACT

This present invention provides a toilet tank lid consisting of Built-in and motion sensor with a rotating fan and an Air freshener console. The built in motion sensor is activated when there is movement across the sensor which then triggers the fan and releases air freshener scent in the bathroom. The fan is seated inside the lid at the top center pulling air from the air freshener console and blowing it out upright therefore its effective in delivering the appropriate scent to eliminate bad odor or smell during and after use of bathroom. This design is not only lightweight, But the convenient and Stylish toilet lid heat/motion sensor air freshener design reduces or eliminates bad odor.

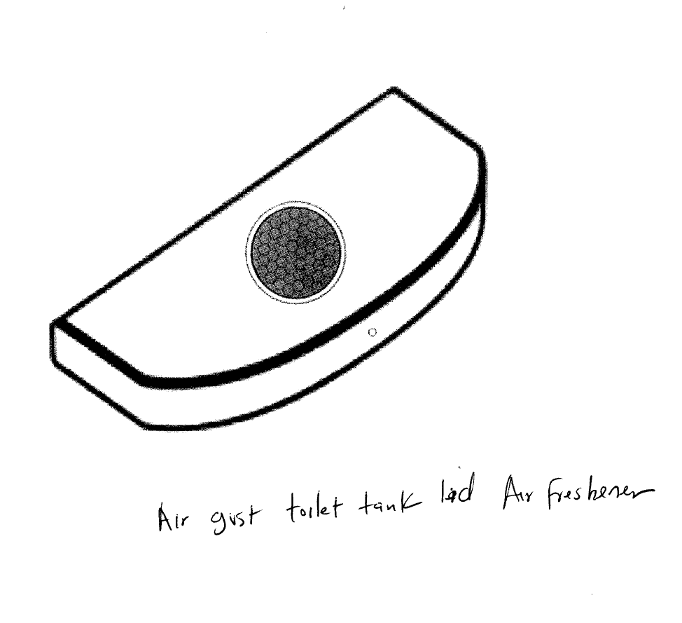

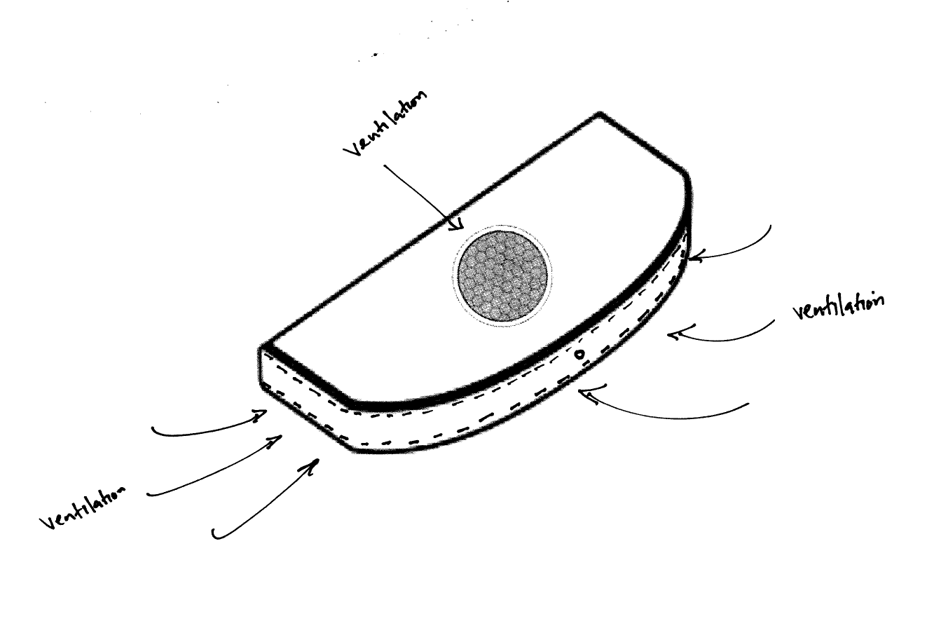

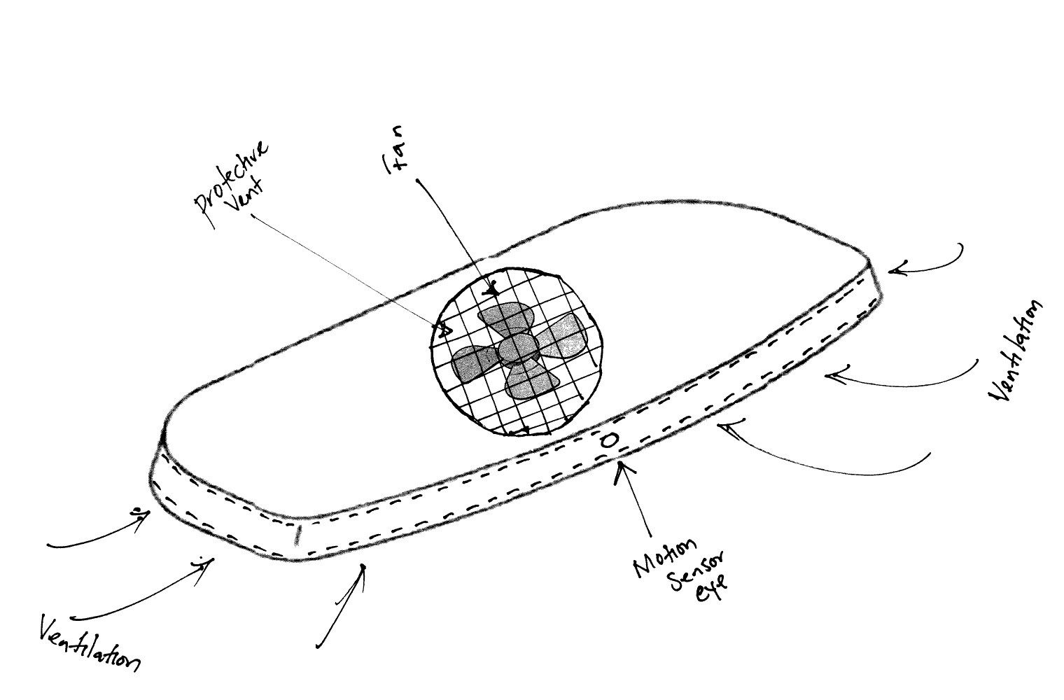

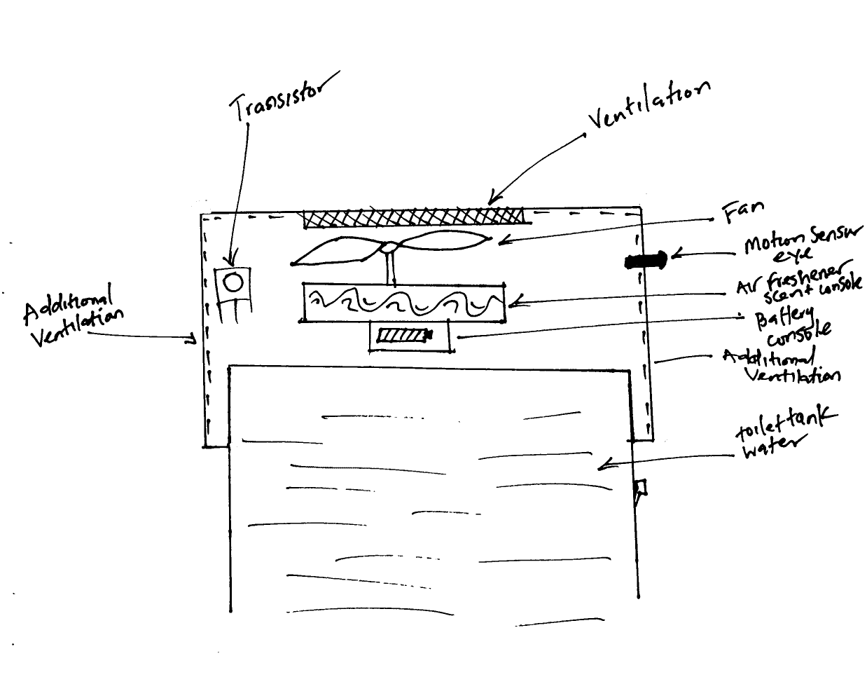

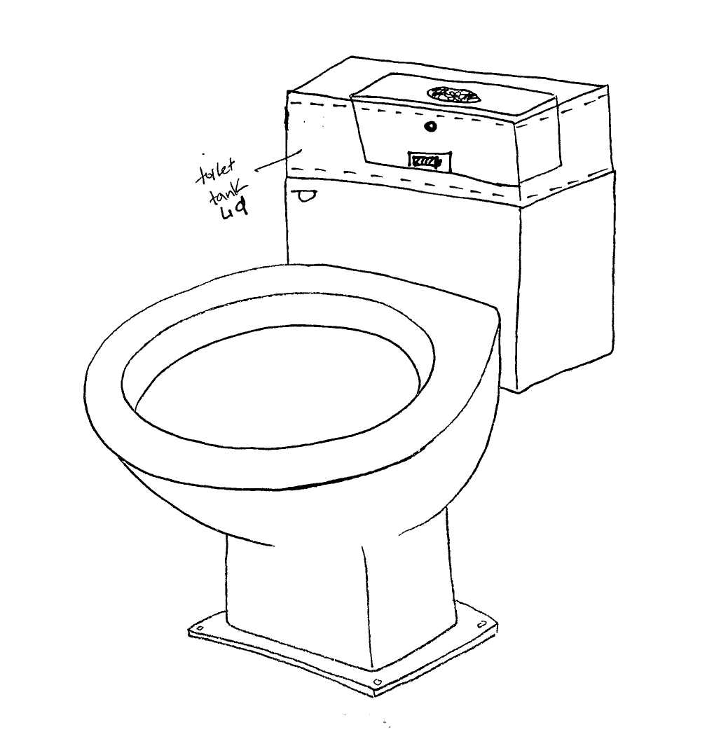

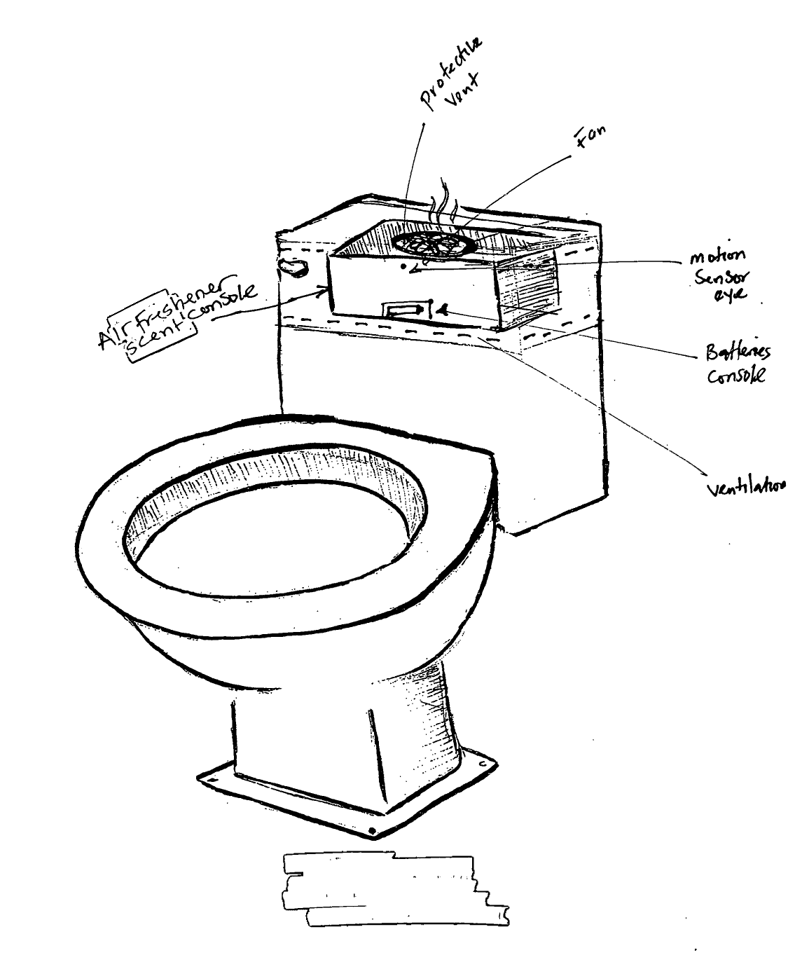

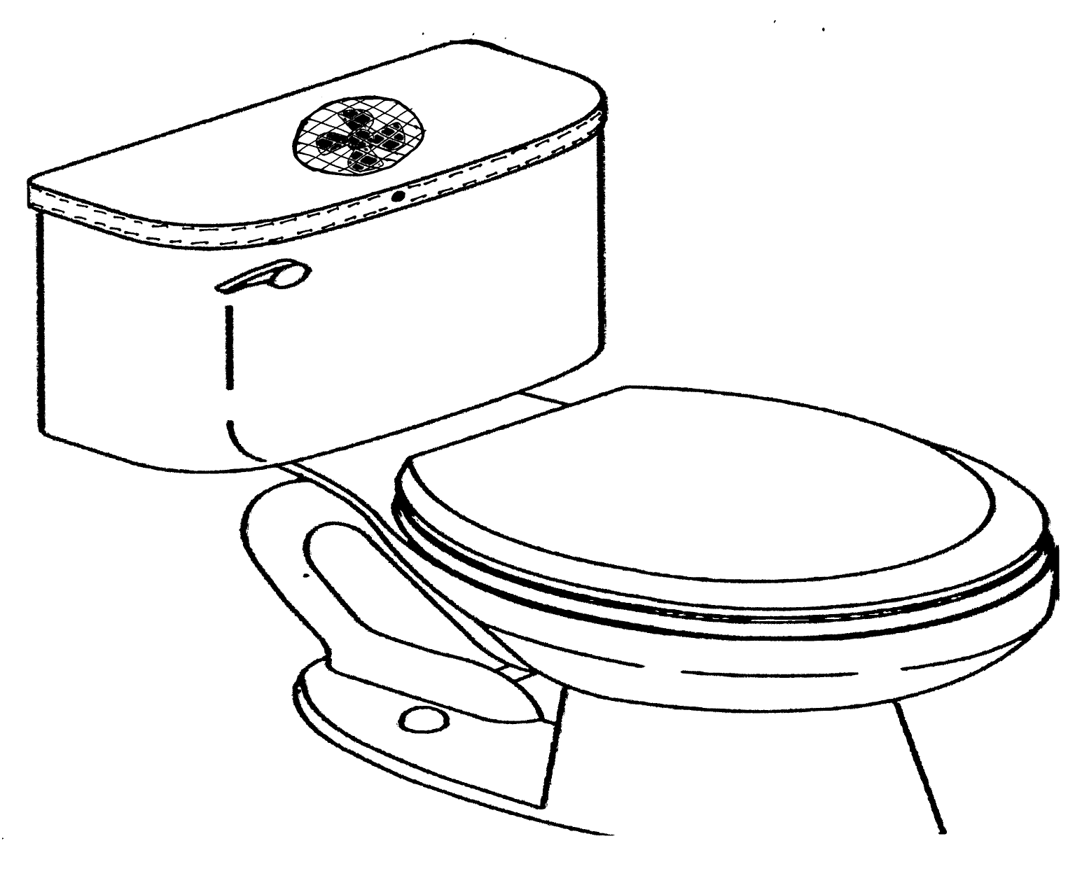

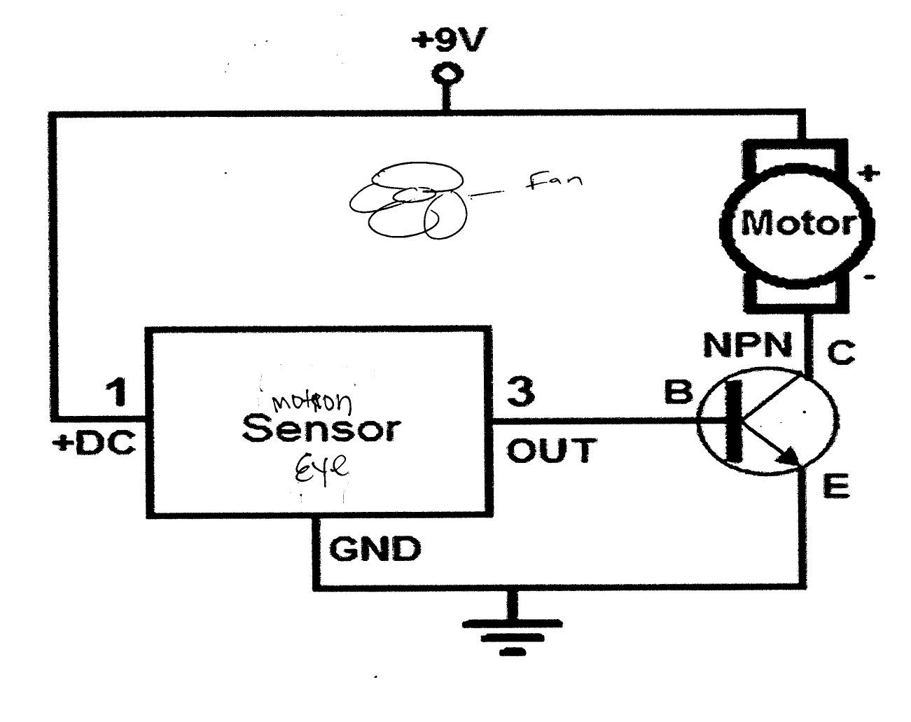

### BACKGROUND OF INVENTION

The present invention relates to toilet air freshener, and more particularly to a toilet tank lid with a built-in motion sensor and rotating fan that dispenses various scent in the bathroom. Common house hold bathroom air freshener are either sprayed or placed in a certain area in the bathroom, which makes the scent fade quickly or circulate in a small area, which is not as effective. This however can result in a bathroom with bad odor constantly. Accordingly it is desired to have a motion Activated air freshener that is built as a toilet tank lid triggered by motion or body heat to dispense the proper scent when and after using the toilet to reduce or eliminate odor. This device may operate in combination with a cartridge containing a fragrance. The refillable cartridge may contain a gel, which includes a fragrance and, upon contact with air, the fragrance is released from the gel due atomization. The refillable cartridge may include a wick where a fragranced cube may be drawn up by a rotating fan in the center of the toilet tank lid and evaporate on exposure to the air. The need for odor control is important with the use of the toilet. Most odors that arise from toilets become quickly distributed above and around the area of the toilet making fragrance dispensing in the immediate area of the toilet fairly effective in masking the odors before they circulate throughout the bathroom. In some cases the odor control fragrance may mask the odor with a stronger scent, in other cases it may contain ingredients designed to combat bacteria. In any event, there is a need to distribute the fragrance as much as possible in as short a time as possible in order to defeat the odors. Accordingly, it may be desirable to have an odor control device built-in to the toilet lid itself and activate the device with use of a motion detection sensor, such as entering the bathroom.

With regard to remote activation of an air freshener due to the change in level in a toilet tank, attention is directed to United States Application Publication No. 2007/020488 which was filed on Mar. 6, 2006 and is entitled“Automated. Remote Bathroom. Air Freshener”. The allowed claim is directed at a remote dispenser that is mounted to the ceiling of a bathroom, and can rotate to spray a surrounding area and includes a reservoir, spray pump, nozzle, drive motor and a signal receiver. A sending unit is mounted in the toilet tank, including an antenna, transmitter, a battery, a level sensor and a float. The float includes a proximity sensor and responds to the water level as it moves up and down by moving up and down a shaft and in doing so causes the transmitter to emit a wireless signal to the remote dispenser. The signal actuates the drive motor and pump for a set period of time. The reference does not disclose a toilet tank lid wit a motion sensor that triggers the fan to dispense air freshener scent. U.S. Pat. No. 4,358,860 issued on Nov. 16, 1982 and is entitled “Automatic Actuator For Air Freshener Dispenser Or The Like For Toilets”. It appears to be directed at a cup that rises and descends with fluctuations in the water level and pivots an actuator arm to automatically dispense d predetermined amount of freshener.

United States Application Publications No. 2008/0272208 entitled “Spraying Device” and No. 2008/0279731 entitled “Device” Disclose a spraying device for sanitizing air, which includes a solenoid.

Prior technology, lack the qualities and benefits of an apparatus and method for dispensing of an air freshener scent in a bathroom, with a toilet lid, built-in motion. sensor, fan and air freshener console to dispense air freshener scent by the act of a detecting motion sensor attached to the toilet tank lid as on unit.

### BRIEF SUMMARY OF INVENTION

The primary advantage of the invention is an apparatus and method for dispensing of an air freshener scent in a bathroom by the act of a toilet tank lid. with a motion sensor and fan built-in as a toilet tank lid. unit.

Another advantage of the invention is for a toilet tank lid that reduces and eliminates bad odor from the bathroom. The motion sensor helps start the air freshener as soon as its senses motion, while the built-in fan helps dispense the air freshener scent properly to the entire bathroom to eliminate odor.

Another advantage of the invention is the need to distribute the fragrance as much as possible in as short a time as possible in order to defeat the odors and another advantage of the invention is no tools are needed for installation.

### OBJECT OF INVENTION

It is the object of the invention to provide toilet tank lids with eliminating bad odor potential, heat/motion sensor to activate the fan that dispenses air freshener and also lightweight.

### DETAILED DESCRIPTION OF INVENTION

This is a toilet tank lid with a center mounted apparatus consisting of a fan and motion sensor with air freshener that can be used in any toilet/bathroom to eliminate bad odor and keep the bathroom/toilet area smelling fresh at all times.

This invention is powered by a 9volt battery which, can last for over one month and half (6 weeks) before the 9volt battery may be changed. The battery sends an electric signal to the motion sensor when movement is made, which then triggers the fan to rotate and dispenses air freshener from the air freshener console located on the top of the toilet lid, therefore spreading air freshener scent through the ventilation across the bathroom. This particular toilet lid air freshener is made out of carbon fiber material making it lightweight, durable and easy to place on top of the toilet tank as well as remove it without any complex manpower. This invention is stylish and can come in various colors of choice. Airgust toilet tank lid air freshener should be a sufficient size and diameter to fit any toilet tank, Airgust toilet tank lid air freshener will be available in various sizes to accommodate different toilets and their size, although Size, diameter of airgust toilet tank air freshener lid may vary depending upon the model of different toilet's weight and size.

### BRIEF DESCRIPTION OF DRAWING

The motion sensor receives signal of movement in the bathroom, which then triggers the fan.

The fan blows air upright therefore grabbing scent from the air freshener console and dispensing air freshener scent accordingly throughout the bathroom.

This device is built and attached to the toilet tank lid. A 9volt battery powers the motion sensor and fan allowing the device to run without any electric plugs or wires making it simple, wireless and safe.

1. A machine for dispensing of an air freshener scent in a bathroom and, more specifically, dispensing such by the act of a toilet tank lid with motion sensor comprising: use or a fan triggered. by the motion sensor on top the toilet tank that activates when the motion sensor receives movement; this device may be one that operates and is powered by batteries; the use of a movement motion to activate the device built, into the toilet tank lid may use a motion sensor; the activating motion sensor eye of the device is attached to the toilet tank lid; the devices may operate in combination with a cartridge containing a fragrance; the fragrance cartridge may be refillable; the fragrance may be a liquid; the fragrance may be a gel; the fragrance may be released upon contact with air due to atomization; the fragrance cartridge may include a wick, fragranced liquid may he drawn up the wick and evaporate on exposure to the air; and vaporization of fragrance may occur due to spraying.
